(19) United States
(12) Patent Application Publication
(10) Pub. No.: US 2023/0088974 A1
(43) Pub. Date: Mar. 23, 2023

(54) METHOD, DEVICE, AND COMPUTER PROGRAM FOR PREDICTING OCCURRENCE OF PATIENT SHOCK USING ARTIFICIAL INTELLIGENCE

(71) Applicant: Spass Inc., Seoul (KR)

(72) Inventors: yong hwan kim, Seoul (KR); Jae Bum LEE, Gimpo-si (KR)

(73) Assignee: Spass Inc., Seoul (KR)

(21) Appl. No.: 18/052,731

(22) Filed: Nov. 4, 2022

Related U.S. Application Data
(63) Continuation of application No. PCT/KR2021/005692, filed on May 6, 2021.

(30) Foreign Application Priority Data
May 6, 2020 (KR) 10-2020-0053564
May 6, 2021 (KR) 10-2021-0058733

(51) Int. Cl. G16H 40/63; G06N 7/01

(52) U.S. Cl. CPC G16H 40/63; G06N 7/01

(57) ABSTRACT

A method, device, and computer program for predicting occurrence of patient shock using artificial intelligence are provided. The method for predicting occurrence of patient shock using artificial intelligence according to various embodiments of the present invention is a method performed by a computing device, the method comprising the steps of: collecting biometric data of a patient; extracting one or more feature values from the collected biometric data; and determining the possibility of occurrence of a medical event with respect to the patient using the extracted one or more feature values.

### CROSS-REFERENCE TO RELATED APPLICATION

The present application is a continuation of International Patent Application No. PCT/KR2021/005692, filed on May 6, 2021, which is based upon and claims the benefit of priority to Korean Patent Application No. 10-2020-0053564, filed on May 6, 2020, and Korean Patent Application No. 10-2021-0058733, filed on May 6, 2021. The disclosures of the above-listed applications are hereby incorporated by reference herein in their entirety.

### TECHNICAL FIELD

Various embodiments of the present invention relate to a method, a device, and a computer program for predicting a patient's shock using artificial intelligence.

### BACKGROUND ART

An intensive care unit is a place for patients who have serious problems in breathing, heartbeat, etc. which are essential functions for maintaining life, and is a place for receiving intensive treatment 24 hours a day, 7 days a week, and 365 days a year.

Therefore, in an intensive care unit, it is necessary to measure and analyze patients' bio-data in real time to avoid missing changes in the statuses of the patients under intensive care, and a variety of equipment is provided to check a critical patient's condition related to disease progression or life maintenance such as a pulse rate, blood pressure, breathing, etc. The patients' conditions are measured through the equipment, and the results are displayed so that a medical worker may check the results.

Intensive care units have a significant impact on the survival and death of patients and are important healthcare providers accounting for 25% of Korean medical expenses. However, intensive care units are very outdated in terms of quality and efficiency of treatment such as a shortage of dedicated medical workers for intensive care, difference in quality between hospitals and regions, high mortality during patient transport, etc.

According to the “2014 (first) intensive care unit adequacy evaluation result” published by the Health Insurance Review and Assessment Service in 2016 in an article of the Hankook Ilbo on May 22, 2018, the average number of hospital beds of intensive care units per intensive care unit specialist in Korea was as much as 44.7, 40.4 in high-level general hospitals, and 48.9 in general hospitals. Due to such a high average number of hospital beds per specialist, there is a problem that it is hard for patients in an intensive care unit to meet a specialist.

As a result, there are cases of death because sepsis is not detected in the early stage. In fact, according to a survey by the Health Insurance Review and Assessment Service, the average mortality rate of adult patients in intensive care units in Korea is 16.9%, 14.3% in high-level hospitals, and 17.4% in general hospitals.

To solve this problem, it has been required to develop a medical event prediction system for preventing the death of a serious patient by determining and providing the probability of a medical event which requires rapid treatment, such as sepsis, to medical workers. However, the medical event prediction system shows low accuracy in prediction, and thus it is difficult to use the medical event prediction system in practice.

### DISCLOSURE

### Technical Problem

The present invention is directed to providing a method, a device, and a computer program for predicting a patient's shock using artificial intelligence, in which one or more feature values are extracted by processing and analyzing various types of bio-data collected from a patient, the probability of a medical event (e.g., sepsis, shock, etc.) occurring in the patient is determined using the one or more feature values, and thus the mortality of serious patients can be reduced by predicting and preventing a medical event leading to the death of a serious patient.

The present invention is also directed to providing a method, a device, and a computer program for predicting a patient's shock using artificial intelligence, in which the probability of a medical event can be accurately predicted using not only data values of a patient's bio-data but also variations of the data values and attributes of a radial graph (e.g., the area between pieces of bio-data, the whole area, the color, the shape, etc. of the graph) generated using the patient's bio-data as factors for determining the probability of the medical event.

Objects of the present invention are not limited to those described above, and other objects which have not been described will be clearly understood by those of ordinary skill in the art from the following descriptions.

### Technical Solution

One aspect of the present invention provides a method of predicting a patient's shock using artificial intelligence by a computing device, the method including collecting bio-data of a patient, extracting one or more feature values from the collected bio-data, and determining a probability of a medical event occurring in the patient using the extracted one or more feature values.

The collecting of the bio-data may include collecting a plurality of pieces of bio-data including at least one of a shock index (SI), respiration (Rr), saturation of percutaneous oxygen (SpO₂), a temperature (Temp), a heart rate (Hr), and a mean arterial pressure (MAP) of the patient, and the extracting of the one or more feature values may include converting each of the plurality of pieces of bio-data into a value within a preset range, generating a radial graph in which each individual axis corresponds to one of the plurality of pieces of bio-data converted into the value within the preset range, and extracting the one or more feature values using the generated radial graph.

The converting of each of the plurality of pieces of bio-data into the value within the preset range may include converting the SI and the Temp of the patient into values within a range of 0 to 1 using SIs and Temps obtained by transforming SIs and Temps of a plurality of patients into a normal distribution and calculating an approximate function for each of Rr, SpO₂, Hr, and MAP of each of the plurality of patients using Bayesian probability distributions of Rr, SO₂, Hr, and MAP when the medical event occurs in the plurality of patients and converting each of the SO₂, the Hr, and the MAP of the patient into a value within a range of 0 to 1 using the calculated approximate function.

The extracting of the one or more feature values using the generated radial graph may include extracting a data value of bio-data collected at a first time point as a feature value, extracting a bio-data variation which is a difference value between the data value of the bio-data collected at the first time point and a data value of bio-data collected for a certain past time period from the first time point as a feature value, and extracting an area between one or more different axes in the generated radial graph as a feature value.

The extracting of the area between the one or more different axes in the generated radial graph as the feature value may include extracting, as feature values, an area between a first axis representing MAP and a second axis disposed adjacent to the first axis and representing Hr, an area between the second axis and a third axis disposed adjacent to the second axis and representing Temp, an area between the third axis and a fourth axis disposed adjacent to the third axis and representing SpO₂, an area between the fourth axis and a fifth axis disposed adjacent to the fourth axis and representing Rr, an area between the fifth axis and a sixth axis disposed adjacent to the fifth axis and representing SI, and an area between the sixth axis and the first axis disposed adjacent to the sixth axis.

The extracting of the bio-data variation as the feature value may include determining the first time point and a plurality of past time points preceding the first time point, determining a plurality of combination pairs of time points including the first time point and the plurality of past time points, calculating a variation of bio-data values corresponding to time points included in each of the plurality of combination pairs, and calculating a feature value of the bio-data variation using the variations each calculated from the plurality of combination pairs.

The extracting of the bio-data variation as the feature value may include setting an approximate function in the form of a quadratic function using the data value of the bio-data collected at the first time point and the data value of the bio-data collected for the certain past time period and calculating a rate of change of the bio-data using the set at least one approximate function and calculating the bio-data variation using the calculated rate of change.

The extracting of the one or more feature values using the generated radial graph may include extracting an image of the generated radial graph as a feature, and the determining of the probability of the medical event may include inputting the image of the radial graph to a trained model to determine the probability of the medical event on the basis of an output of the trained model.

The extracting of the image of the generated radial graph as the feature may include calculating a bio-data variation which is a difference value between a data value of bio-data collected at a first time point and a data value of bio-data collected for a certain past time period from the first time point, showing the calculated bio-data variation on the generated radial graph, and extracting an image of the radial graph showing the calculated bio-data variation as a feature.

The generating of the radial graph may include connecting each of the SI, the Rr, the SpO₂, the Temp, the Hr, and the MAP to the adjacent bio-data to form a closed curve and determining a color inside the formed closed curve in accordance with an area inside the formed closed curved, and the extracting of the one or more feature values using the generated radial graph may include extracting the determined color inside the closed curve as a feature value.

The determining of the probability of the medical event may include training an artificial intelligence model using bio-data of a plurality of patients as training data and extracting result data about the probability of the medical event using the extracted one or more feature values as input values for the trained artificial intelligence model and determining the probability of the medical event using the extracted result data, and the training of the artificial intelligence model may include labeling each of bio-data collected at a time point at which the medical event has occurred and bio-data collected for a certain past time period from the time point at which the medical event has occurred among the bio-data of the plurality of patients with information about whether the medical event has occurred, a type of medical event, and a time point at which the bio-data has been collected to generate the training data and training the artificial intelligence model in accordance with supervised learning using the generated training data.

The artificial intelligence model may include a plurality of artificial intelligence models, and the determining of the probability of the medical event using the extracted result data may include extracting one piece of result data about the probability of the medical event using any one of the plurality of artificial intelligence models and determining the probability of the medical event using the extracted one piece of result data or extracting two or more pieces of result data about the probability of the medical event using two or more of the plurality of artificial intelligence models and aggregating the extracted two or more pieces of result data to determine the probability of the medical event.

Another aspect of the present invention provides a device for predicting a patient's shock using artificial intelligence, the device including a processor, a network interface, a memory, and a computer program loaded into the memory and executed by the processor. The computer program includes an instruction for collecting bio-data of a patient, an instruction for extracting one or more feature values from the collected bio-data, and an instruction for determining a probability of a medical event occurring in the patient using the extracted one or more feature values.

Another aspect of the present invention provides a computer program recorded on a computer-readable recording medium to perform, in combination with a computing device, collecting bio-data of a patient, extracting one or more feature values from the collected bio-data, and determining a probability of a medical event occurring in the patient using the extracted one or more feature values.

Other details of the present invention are included in the detailed description and drawings.

### Advantageous Effects

According to various embodiments of the present invention, it is possible to extract one or more feature values by processing and analyzing various types of bio-data collected from a patient, and determine the probability of a medical event (e.g., sepsis, shock, etc.) occurring in the patient using the one or more feature values, and thus the mortality of serious patients can be reduced by predicting and preventing a medical event leading to the death of a serious patient.

Also, it is possible to accurately predict the probability of a medical event using not only data values of a patient's bio-data but also variations of the data values and attributes of a radial graph (e.g., the area between pieces of bio-data, the whole area, the color, the shape, etc. of the graph) generated using the patient's bio-data as factors for determining the probability of the medical event.

Effects of the present invention are not limited to those described above, and other effects which have not been described will be clearly understood by those of ordinary skill in the art from the following descriptions.

### DESCRIPTION OF DRAWINGS

FIG. 1 is a diagram of a system for predicting a patient's shock using artificial intelligence according to an embodiment of the present invention.

FIG. 2 is a diagram showing a hardware configuration of a device for predicting a patient's shock using artificial intelligence according to another embodiment of the present invention.

FIG. 3 is a flowchart illustrating a method of predicting a patient's shock using artificial intelligence according to still another embodiment of the present invention.

FIG. 4 is a flowchart illustrating a method of extracting a feature value on the basis of a radial graph according to various embodiments.

FIG. 5 illustrates a radial graph generated by a device for predicting a patient's shock using artificial intelligence according to various embodiments.

FIGS. 6A-6B illustrates a set of bio-data graphs over time to describe a method of extracting variations of bio-data as one or more feature values according to various embodiments.

FIG. 7 is a set of graphs showing a bio-data variation depending on whether there is a medical event according to various embodiments.

FIG. 8 is a diagram showing a configuration for calculating the area between pieces of bio-data corresponding to different axes as one or more feature values according to various embodiments.

FIG. 9 illustrates a radial graph in which bio-data variations are shown according to various embodiments.

FIG. 10 is a flowchart of a method of training an artificial intelligence model using training data and determining the probability of a medical event using the trained artificial intelligence model according to various embodiments.

FIG. 11 is a diagram illustrating an implementation form of an artificial intelligence model that is applicable to various embodiments.

### MODES OF THE INVENTION

Advantages and features of the present invention and a method of achieving them will become apparent from embodiments which will be described in detail below with reference to the accompanying drawings. However, the present invention is not limited to the embodiments disclosed below and may be implemented in various forms. The embodiments are only provided to make the disclosure of the present invention complete and fully inform those skilled in the technical field to which the present invention pertains of the scope of the present invention. The present invention is only defined by the scope of the claims.

Terminology used herein is for the purpose of describing embodiments only and is not intended to limit the present invention. As used herein, the singular forms include the plural forms as well unless the context clearly indicates otherwise. The terms “comprises” and/or “comprising” do not preclude the presence or addition of one or more components other than stated components. Throughout the specification, like numbers refer to like components, and “and/or” includes any one or all possible combinations of stated components. Although “first,” “second,” etc. are used to describe various components, the components are not limited by the terms. These terms are used to distinguish one component from other components. Accordingly, it is apparent that a first component described below may be a second component without departing from the technical spirit of the present invention.

Unless otherwise defined, all terms (including technical and scientific terms) may have meanings generally understood by those skilled in the technical field to which the present invention pertains. Also, unless clearly defined, all terms defined in generally used dictionaries are not to be ideally or excessively interpreted.

The term “unit” or “module” used herein means a software or hardware component, such as a field-programmable gate array (FPGA) or an application-specific integrated circuit (ASIC), and a “unit” or “module” performs certain roles. However, a “unit” or “module” is not limited to software or hardware. A “unit” or “module” may be in an addressable storage medium or may be configured to run on one or more processors. Therefore, as an example, a “unit” or “module” includes components such as software components, object-oriented software components, class components, and task components, as well as processes, functions, attributes, procedures, subroutines, segments of program code, drivers, firmware, micro-code, circuits, data, databases, data structures, tables, arrays, and variables. Functions provided in components and “units” or “modules” may be combined into a smaller number of components and “units” or “modules” or subdivided into additional components and “units” or “modules.”

Spatially relative terms, such as “below,” “beneath,” “lower,” “above,” “upper,” etc., as illustrated in the drawings, may be used to facilitate the description of relationships between a component and other components. The spatially relative terms should be understood as terms that include different directions of the component in use or operation in addition to the direction illustrated in the drawings. For example, when a component illustrated in a drawing is reversed, another component described to be disposed “below” or “beneath” the component may be disposed “above” the component. Accordingly, the exemplary term “below” may include both upward and downward directions. A component may be directed in another direction, and the spatially relative terms may be interpreted accordingly.

In the specification, a computer is any type of hardware device including at least one processor and may be understood as encompassing software elements operating in the corresponding hardware device according to embodiments. For example, examples of a computer may be understood as including but not limited to all of a smartphone, a tablet personal computer (PC), a desktop, a laptop, and a user client and applications running on each of the devices.

In this specification, a patient is assumed to be in an intensive care unit, and clinical treatment provided in the intensive care unit is assumed to be universal, that is, all patients in intensive care units are assumed to receive universal treatment from medical workers.

Also, a change in bio-data of a patient is detected using the bio-data of the patient, and the probability of a medical event is determined in accordance with the pattern of change. Since the determination depends on a change in bio-data of a patient, patients having overly irregular changes in the bio-data (e.g., patients with heart diseases, such as arrhythmias, cardiogenic diseases, etc., and the like) may be excluded from targets.

Hereinafter, embodiments of the present invention will be described in detail with reference to the accompanying drawings.

Each step described herein is described as being performed by a computer, but the subject of each step is not limited thereto, and at least some of the steps may be performed in different devices according to embodiments.

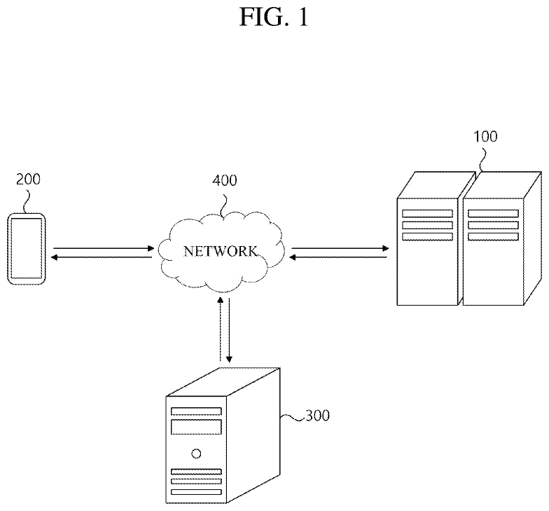

FIG. 1 is a diagram of a system for predicting a patient's shock using artificial intelligence according to an embodiment of the present invention.

Referring to FIG. 1, the system for predicting a patient's shock using artificial intelligence may include a shock prediction device 100, an external terminal 200, and an external server 300. Here, the system for predicting a patient's shock using artificial intelligence shown in FIG. 1 is in accordance with an embodiment. The components are not limited to the embodiment shown in FIG. 1 and may be added, changed, or removed as necessary.

According to an embodiment, the shock prediction device 100 may extract one or more feature values from bio-data of a patient and determine the probability of a medical event (e.g., sepsis) occurring in the patient using the extracted one or more feature values. For example, the shock prediction device 100 may extract result data about the probability of the medical event using the one or more feature values as inputs for artificial intelligence trained in advance and determine the probability of the medical event occurring in the patient using the extracted result data. However, the present invention is not limited thereto.

According to various embodiments, the shock prediction device 100 may be communicably connected to the external terminal 200 through a network 400 and may provide information on the probability of a medical event occurring in the patient which is determined on the basis of the bio-data of the patient to the external terminal 200 for taking care of the patient.

Here, the probability of the medical event may be provided in the form of a percentage (%). When there are a plurality of medical events of which the probabilities will be determined, the probability of each of the plurality of medical events may be separately determined and provided, but the present invention is not limited thereto.

Also, when the probability of the medical event occurring in the patient is determined to be greater than or equal to a criterion on the basis of the bio-data of the patient, the shock prediction device 100 may provide a warning message recommending action on the medical event and guidance on a method of handling the medical event and determine the form and intensity of the output warning message in accordance with the probability of the medical event.

In various embodiments, the shock prediction device 100 may be connected to the external server 300 through the network 400 and may provide bio-data of each of a plurality of patients and data about the probability of a medical event determined on the basis of the bio-data to the external server 300 and store and manage the data in the external server 300.

According to an embodiment, the external terminal 200 may be communicably connected to the shock prediction device 100 through the network 400 and may collect and transmit bio-data of a patient to the shock prediction device 100 and receive information on the probability of a medical event occurring in the patient in response to the transmitted bio-data.

According to various embodiments, the external terminal 200 may include a sensor module which is attached to and installed on at least a part of the patient's body to collect sensor data of the patient, or collect bio-data of the patient from a sensor module separately provided outside the external terminal 200. The external terminal 200 may transmit the bio-data of the patient measured by the sensor module provided thereto or the bio-data of the patient collected from the external sensor module to the shock prediction device 100.

Also, the external terminal 200 may have a display in at least a part thereof and output the bio-data of the patient or information on the probability of a medical event based on the bio-data through the display. For example, the external terminal 200 may be a bio-data output device, such as a bedside monitoring device in an intensive care unit but is not limited thereto. The external terminal 200 may be a portable terminal such as a smartphone of a medical worker who takes care of the patient.

According to an embodiment, the external server 300 may be communicably connected to the shock prediction device 100 through the network 400 and provide various types of information (e.g., bio-data of a plurality of patients, bio-data of the patient whose probability of a medical event will be determined for a certain past time period, etc.) required for the shock prediction device 100 to determine the probability of a medical event.

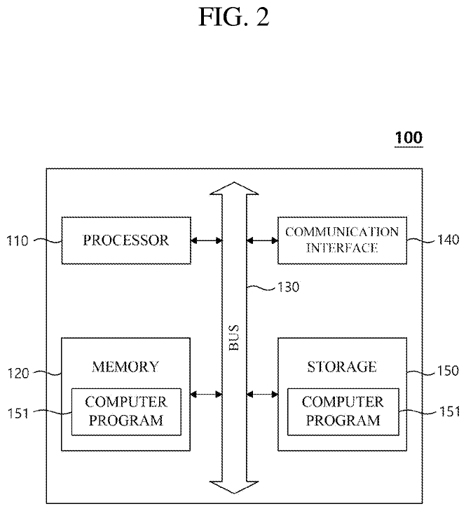

Also, the external server 300 may receive and store various types of information which is generated when the shock prediction device 100 determines the probability of a medical event occurring in the patient. For example, the external server 300 may be, but is not limited to, a storage server that is separately provided outside the shock prediction device 100 to store and manage a large amount of data. A hardware configuration of the shock prediction device 100 for performing a method of predicting a patient's shock using artificial intelligence will be described below with reference to FIG. 2.

FIG. 2 is a diagram showing a hardware configuration of a device for predicting a patient's shock using artificial intelligence according to another embodiment of the present invention.

Referring to FIG. 2, the shock prediction device 100 (hereinafter a “computing device 100”) according to another embodiment of the present invention may include at least one processor 110, a memory 120 into which a computer program 151 executed by the processor 110 is loaded, a bus 130, a communication interface 140, and a storage 150 for storing the computer program 151. In FIG. 2, only components related to embodiments of the present invention are shown. Accordingly, those skilled in the technical field to which the present invention pertains may appreciate that other general-purpose components may be included in addition to the components shown in FIG. 2.

The processor 110 controls overall operations of each component of the computing device 100. The processor 110 may include a central processing unit (CPU), a micro-processor unit (MPU), a micro-controller unit (MCU), a graphics processing unit (GPU), or any form of processor well known in the technical field of the present invention.

The processor 110 may perform computation for at least one application or program for performing methods according to embodiments of the present invention, and the computing device 100 may include at least one processor.

According to various embodiments, the processor 110 may further include a random access memory (RAM) (not shown) and read-only memory (ROM) (not shown) which temporarily and/or permanently store signals (or data) processed in the processor 110. The processor 110 may be implemented in the form of a system on chip (SoC) including at least one of a GPU, a RAM, and a ROM.

The memory 120 stores various types of data, commands, and/or information. The memory 120 may load the computer program 151 from the storage 150 to perform methods/operations according to various embodiments of the present invention. When the computer program 151 is loaded into the memory 120, the processor 110 may perform the methods/operations by executing one or more instructions constituting the computer program 151. The memory 120 may be implemented as a volatile memory, such as a RAM, but the technical scope of the present disclosure is not limited thereto.

The bus 130 provides a communication function between components of the computing device 100. The bus 130 may be implemented in various forms of buses such as an address bus, a data bus, a control bus, etc.

The communication interface 140 supports wired or wireless Internet communication of the computing device 100. Also, the communication interface 140 may support various communication methods other than Internet communication. To this end, the communication interface 140 may include communication modules well known in the technical field of the present invention. In some embodiments, the communication interface 140 may be omitted.

The storage 150 may non-temporarily store the computer program 151. When a process of determining the probability of a medical event occurring in a patient is performed through the computing device 100, the storage 150 may store various types of information required for providing the process of determining the probability of a medical event occurring in a patient. For example, when a process of determining the probability of a medical event occurring in a first patient is performed by the computing device 100, the storage 150 may receive bio-data of the first patient from the external server 300 which stores and manages bio-data of a plurality of patients, and may store the bio-data of the first patient.

The storage 150 may include a non-volatile memory, such as a ROM, an erasable programmable ROM (EPROM), an electrically erasable programmable ROM (EEPROM), a flash memory, etc., a hard disk, a detachable disk, or any form of computer-readable recording medium which is well known in the technical field to which the present invention pertains.

The computer program 151 may include one or more instructions that cause the processor 110 to perform the methods/operations according to various embodiments of the present invention when the instructions are loaded into the memory 120. In other words, the processor 110 may perform the methods/operations according to various embodiments by executing the one or more instructions.

According to an embodiment, the computer program 151 may include one or more instructions for performing a method of predicting a patient's shock using artificial intelligence, the method including an operation of collecting bio-data of a patient, an operation of extracting one or more feature values from the collected bio-data, and an operation of determining the probability of a medical event occurring in the patient using the extracted one or more feature values.

Steps of a method or algorithm described regarding an embodiment of the present invention may be directly implemented as hardware, implemented as a software module executed by hardware, or implemented as a combination of the hardware and software module. The software module may be on a RAM, a ROM, an EPROM, an EEPROM, a flash memory, a hard disk, a detachable disk, a compact disc (CD)-ROM, or any type of computer-readable recording medium which is well known in the technical field to which the present invention pertains.

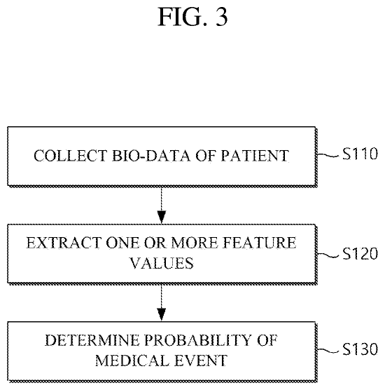

Components of the present invention may be implemented as a program (or an application) and stored in a medium to be executed in combination with a computer which is hardware. Components of the present invention may be implemented with software programming or software modules. Similarly, embodiments may be implemented with a programming or scripting language, such as C, C++, Java, assembler, etc., to include various algorithms implemented as data structures, processes, routines, or combinations of other programming elements. Functional aspects may be implemented by an algorithm that is executed in one or more processors. A method of predicting a patient's shock using artificial intelligence by the computing device 100 will be described below with reference to FIG. 3.

FIG. 3 is a flowchart illustrating a method of predicting a patient's shock using artificial intelligence according to another embodiment of the present invention.

Referring to FIG. 3, in operation S110, the computing device 100 may collect bio-data of a patient. For example, the computing device 100 may include a sensor module which is attached to and installed on at least a part of the patient's body to collect sensor data of the patient, or may collect bio-data of the patient from the external terminal 200 which may collect the bio-data of the patient from a sensor module separately provided outside the external terminal 200.

The bio-data of the patient may include at least one of a shock index (SI), respiration (Rr), saturation of percutaneous oxygen (SpO₂), a temperature (Temp), a heart rate (Hr), and a mean arterial pressure (MAP) of the patient but is not limited thereto. The bio-data of the patient may be various types of bio-data for understanding the patient's condition.

According to various embodiments, the bio-data of the patient whose probability of a medical event will be determined may be directly input to the computing device 100 by a medical worker in charge of the patient. However, the present invention is not limited thereto, and various methods of collecting bio-data of a patient are applicable.

According to various embodiments, the computing device 100 may collect the bio-data of the patient at preset unit time intervals (e.g., one hour).

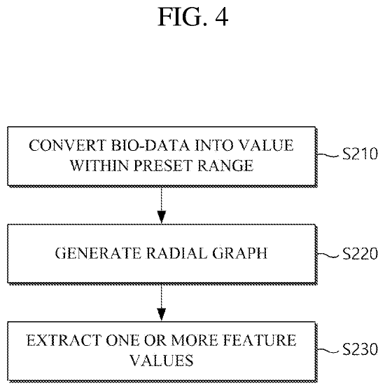

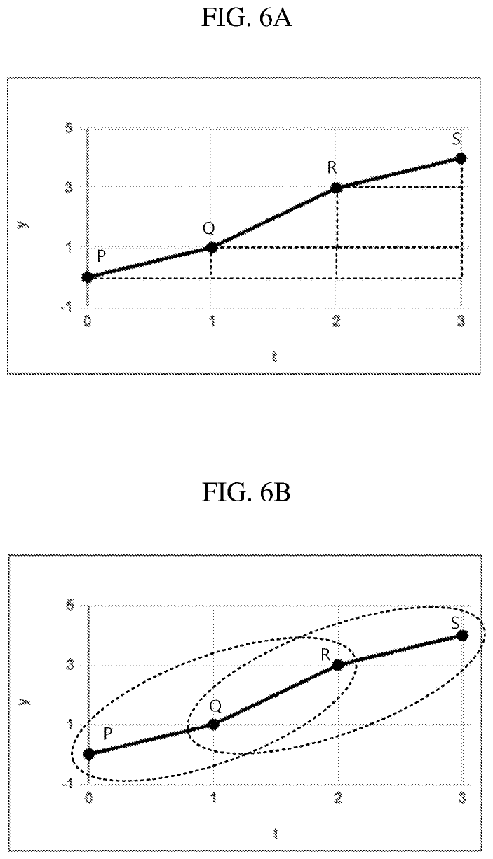

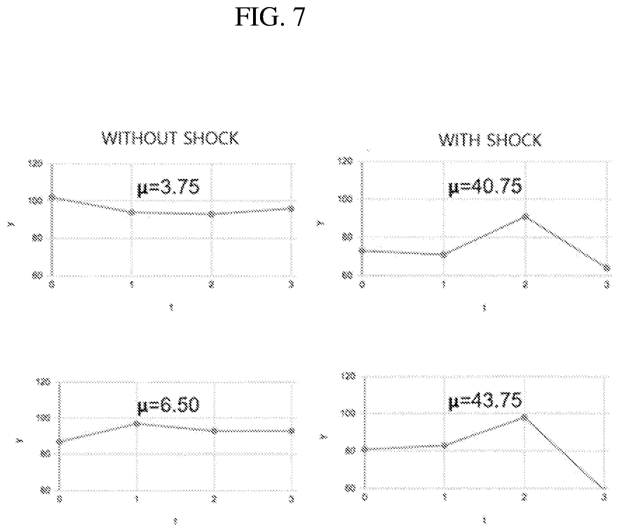

In operation S120, the computing device 100 may extract one or more feature values from the bio-data of the patient. A method of extracting one or more feature values by the computing device 100 will be described below with reference to FIGS. 4 to 13.

FIG. 4 is a flowchart illustrating a method of extracting a feature value on the basis of a radial graph according to various embodiments.

Referring to FIG. 4, in operation S210, the computing device 100 may convert each of a plurality of pieces of bio-data (e.g., SI, Rr, SpO₂, Temp, Hr, and MAP) of a patient into a value within a preset range.

Here, the preset range may be in the of 0 to 1. The values may be set in advance by a manager or medical worker who performs the method of predicting a patient's shock using artificial intelligence but are not limited thereto.

According to various embodiments, the computing device 100 may convert the bio-data into a value within the range of 0 to 1 using a normal distribution in accordance with features of the bio-data or convert the bio-data into a value within the range of 0 to 1 through approximate function estimation in accordance with a probability distribution.

First, the computing device 100 may generate an SI normal distribution diagram by transforming SIs of a plurality of patients into a normal distribution and convert an SI of a patient into a value within the range of 0 to 1 by inserting the SI of the patient whose probability of a medical event will be determined to the SI normal distribution diagram. However, the present invention is not limited thereto, and the computing device 100 may not directly generate the SI normal distribution diagram using the SIs of the plurality of patients but may convert the SI of the patient into a value within the range of 0 to 1 using an SI normal distribution diagram (e.g., an externally generated SI distribution diagram) generated by transforming SIs of a plurality of patients into a normal distribution in advance.

Here, the SI converted into the value within the range of 0 to 1 is a value representing likelihood. The SI may be a value representing a probability that the corresponding SI will be measured when the medical event occurs in the patient, but is not limited thereto.

Also, the computing device 100 may generate a Temp normal distribution diagram by transforming Temps of a plurality of patients into a normal distribution and convert the Temp of the patient into a value within the range of 0 to 1 by inserting the Temp of the patient whose probability of the medical event will be determined to the Temp normal distribution diagram. However, the present invention is not limited thereto, and the computing device 100 may not directly generate the Temp normal distribution diagram using the Temps of the plurality of patients but may convert the Temp of the patient into a value within the range of 0 to 1 using a Temp normal distribution diagram (e.g., an externally generated Temp distribution diagram) generated by transforming Temps of a plurality of patients into a normal distribution in advance.

Here, the Temp converted into the value within the range of 0 to 1 is a value representing a probability that the corresponding Temp will be measured when the medical event occurs in the patient, but is not limited thereto.

Meanwhile, the computing device 100 may calculate Bayesian probability values of MAPs when the medical event occurs in a plurality of patients and calculate a preset first approximate function using a distribution of the calculated Bayesian probability values of the MAPs. The computing device 100 may convert the MAP of the patient into a value within the range of 0 to 1 by inserting the MAP of the patient whose probability of the medical event will be determined to the first approximate function. However, the present invention is not limited thereto, and the computing device 100 may not directly generate the Bayesian probability distribution of MAPs using the MAPs of the plurality of patients but may convert the MAP of the patient into a value within the range of 0 to 1 using a Bayesian probability distribution of MAPs (e.g., an externally generated Bayesian probability distribution of MAPs) generated using Bayesian probability values of MAPs of a plurality of patients in advance.

Here, the MAP converted into the value within the range of 0 to 1 is a value representing a probability that the corresponding MAP will be measured when the medical event occurs in the patient, but is not limited thereto.

The computing device 100 may calculate Bayesian probability values of SpO₂ when the medical event occurs in a plurality of patients and calculate a preset second approximate function using a distribution of the calculated Bayesian probability values of the SpO₂. The computing device 100 may convert the SpO₂ of the patient into a value within the range of 0 to 1 by inserting the SpO₂ of the patient whose probability of the medical event will be determined to the second approximate function. However, the present invention is not limited thereto, and the computing device 100 may not directly generate the Bayesian probability distribution of SpO₂ using the SpO₂ of the plurality of patients but may convert the SpO₂ of the patient into a value within the range of 0 to 1 using a Bayesian probability distribution of SpO₂ (e.g., an externally generated Bayesian probability distribution of SpO₂) generated using Bayesian probability values of SpO₂ of a plurality of patients in advance.

Here, the SpO₂ converted into the value within the range of 0 to 1 is a value representing a probability that the corresponding SpO₂ will be measured when the medical event occurs in the patient, but is not limited thereto.

The computing device 100 may calculate Bayesian probability values of Hr when the medical event occurs in a plurality of patients and calculate a preset third approximate function using a distribution of the calculated Bayesian probability values of the Hr. The computing device 100 may convert the Hr of the patient into a value within the range of 0 to 1 by inserting the Hr of the patient whose probability of the medical event will be determined to the second approximate function. However, the present invention is not limited thereto, and the computing device 100 may not directly generate the Bayesian probability distribution of Hr using the Hr of the plurality of patients but may convert the Hr of the patient into a value within the range of 0 to 1 using a Bayesian probability distribution of Hr (e.g., an externally generated Bayesian probability distribution of Hr) generated using Bayesian probability values of Hr of a plurality of patients in advance.

Here, the Hr converted into the value within the range of 0 to 1 is a value representing a probability that the corresponding Hr will be measured when the medical event occurs in the patient, but is not limited thereto.

Also, the computing device 100 may calculate Bayesian probability values of Rr when the medical event occurs in a plurality of patients and calculate a preset fourth approximate function using a distribution of the calculated Bayesian probability values of the Rr. The computing device 100 may convert the Rr of the patient into a value within the range of 0 to 1 by inserting the Rr of the patient whose probability of the medical event will be determined to the fourth approximate function. However, the present invention is not limited thereto, and the computing device 100 may not directly generate the Bayesian probability distribution of Rr using the Rr of the plurality of patients but may convert the Rr of the patient into a value within the range of 0 to 1 using a Bayesian probability distribution of Rr (e.g., an externally generated Bayesian probability distribution of Rr) generated using Bayesian probability values of Rr of a plurality of patients in advance.

Here, the Rr converted into the value within the range of 0 to 1 is a value representing a probability that the corresponding Rr will be measured when the medical event occurs in the patient, but is not limited thereto.

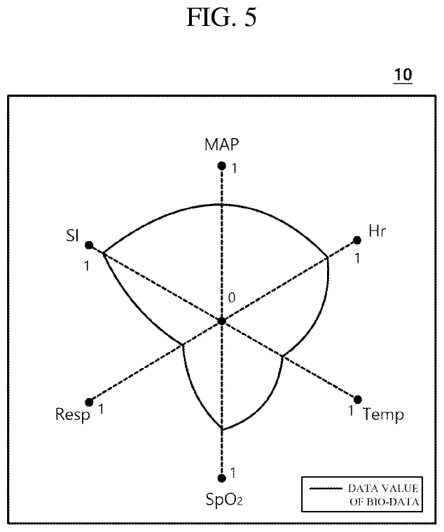

In other words, the computing device 100 may convert the SI and Temp of the patient into a value within the range of 0 to 1 using a normal distribution and convert the Rr, the SpO₂, the Hr, and the MAP into a value within the range of 0 to 1 through estimation based on an approximate function of a probability distribution. In operation S220, the computing device 100 may generate a radial graph in which each individual axis corresponds to one of the plurality of pieces of bio-data converted into the value within the preset range (e.g., a value within the range of 0 to 1). A radial graph generation method performed by the computing device 100 will be described below with reference to FIG. 5.

FIG. 5 illustrates a radial graph generated by a device for predicting a patient's shock using artificial intelligence according to various embodiments.

Referring to FIG. 5, when bio-data collected form a user is SI, Resp, SpO₂, Temp, Hr, and MAP, the computing device 100 may generate a radial graph in which each of SI, Resp, SpO₂, Temp, Hr, and MAP corresponds to an individual axis. Since an SI, Resp, SpO₂, a Temp, an Hr, and a MAP converted into a value within the range of 0 to 1 are used, the computing device 100 may generate a radial graph in which each axis has a minimum value of 0 and a maximum value of 1, and may display a bio-data value corresponding to each axis.

The computing device 100 may arrange the plurality of axes so that the axes have the same angle therebetween. For example, since bio-data collected from the patient is SI, Resp, SpO₂, Temp, Hr, and MAP, that is, six types of bio-data, the computing device 100 may set the angle between the axes to 60° so that the axes may have the same angle therebetween. However, the angle between axes is not limited thereto.

Although it is shown that the computing device 100 sequentially arranges Hr, Temp, SpO₂, Resp, and SI clockwise on the basis of MAP, the order of bio-data arrangement shown in FIG. 5 is only exemplary. The order of bio-data arrangement is not limited thereto and may be variously set by a medical worker and the like who monitors the patient's condition in person.

According to various embodiments, the computing device 100 may construct each individual axis with one of SI, Resp, SO₂, Temp, Hr, and MAP and separately display data values of SI, Resp, SO₂, Temp, Hr, and MAP converted into values within the preset range on the constructed axes. Here, each data value may be reversed and displayed on the radial graph.

Each of the plurality of pieces of bio-data converted into a value within the range of 0 to 1 by the computing device is a probability value, which represents the probability that the data value of the corresponding bio-data will be measured when a medical event occurs in the patient. The closer to 1, the higher the probability that the medical event will occur in the patient. For example, when the medical event is a shock, such as sepsis, it may be determined that a shock has occurred at a MAP of less than 65. Here, the preset first approximate function (approximate MAP function) may have a value of 1 at a time point at which the MAP is 65 and drop to 0.25 or less when the MAP is 65 or more (up to about 120).

When a shock, such as sepsis, occurs, the Hr of the patient increases. For example, the preset third approximate function (Hr approximate function) may be set to the lowest value within the range of a mean heart rate of a human and may increase with an increase in the Hr.

In other words, when a radial graph is generated using the data value without change, the size of the radial graph is reduced with a low probability of the medical event occurring in the patient and increased with a high probability of the medical event. At normal times when no medical event occurs, the graph is very small, and thus it is difficult to monitor data other than medical events. This may cause a problem that it is somewhat inconvenient to quickly and easily understand the patient's condition.

To overcome this, the computing device 100 reverses each of the plurality of pieces of bio-data converted into a value within the range of 0 to 1 to convert a probability (e.g., q) that the data value of the corresponding piece of bio-data will be measured when the medical event occurs in the patient into a probability (e.g., 1-q) that the data value of the corresponding piece of bio-data will be measured when no medical event occurs in the patient and generates a radial graph. Accordingly, the size of the radial graph may increase when the probability of the medical event occurring in the patient is low, and may decrease when the probability of the medical event is high. However, the present invention is not limited thereto, and the computing device 100 may reverse a plurality of pieces of bio-data converted into values within the range of 0 to 1 in accordance with a type of medical event of which the probability will be determined and use the reversed data or may selectively reverse only a specific piece of bio-data among a plurality of pieces of bio-data and use the reversed data.

According to various embodiments, the computing device 100 may form a closed curve by connecting SI, Resp, SpO₂, Temp, Hr, and MAP each corresponding to the axes on the radial graph to adjacent bio-data (data values). For example, the computing device 100 may form a closed curve by connecting the value of MAP, which is a base on the radial graph, to the values of SI and Hr adjacent to MAP, connecting the value of Hr to the value of Temp, connecting the value of Temp to the value of SpO₂, connecting the value of SpO₂ to the value of Resp, and connecting the value of Resp to the value of SI.

According to various embodiments, the computing device 100 may determine a color inside the closed curve formed on the radial graph in accordance with the internal area of the closed curve. For example, the computing device 100 may determine that the color inside the closed curve is black when the internal area of the closed curve is less than a first size (e.g., 0.4), red when the internal area is greater than or equal to the first size and less than a second size (e.g., 0.7), blue when the internal area is greater than or equal to the second size and less than a third size (e.g., 0.8), and green when the internal area is greater than or equal to the third size. However, the present invention is not limited thereto, and the computing device 100 may separately determine colors between the axes in accordance with the areas between the axes on the radial graph.

Referring back to FIG. 4, in operation S230, the computing device 100 may extract one or more feature values for determining the probability of a medical event occurring in the patient using the radial graph.

According to various embodiments, as a feature value, the computing device 100 may extract the data value (e.g., the value of bio-data converted into a value within the range of 0 to 1) of bio-data collected at a first time point that is a current time point at which the probability of the medical event occurring in the patient will be determined.

According to various embodiments, as a feature value, the computing device 100 may extract a bio-data variation which is a difference value between the data value of the bio-data collected at the first time point and the data value of bio-data collected for a certain past time period from the first time point (e.g., three hours immediately before the first time point).

According to various embodiments, the computing device 100 may calculate a bio-data variation using the following method.

For example, the computing device 100 may determine a first time point which is a base time point and one or more past time points having preset time intervals from the first time point (e.g., a second time point which is one hour before the first time point, a third time point which is two hours before the first time point, a fourth time point which is three hours before the first time point, etc.).

The computing device 100 may determine various combination pairs of the first time point and a plurality of time points including one or more past time points and calculate variations in bio-data between time points corresponding to the determined combination pairs. With respect to each piece of bio-data, the computing device 100 may calculate a parameter representing a bio-data variation using variations between pieces of bio-data of time points corresponding to combination pairs of different time points.

For example, when there is a combination pair of the first time point and the second time point, the difference between a bio-data value of the first time point and a bio-data value of the second time point may be calculated as a variation that the corresponding combination pair represents, and a parameter representing the bio-data variation may be calculated through calculation (e.g., addition, average, etc.) with variations calculated from other combination pairs.

According to various embodiments, the computing device 100 may set an approximate function in the form of a quadratic function using the data value of the 5 bio-data collected at the first time point and data values of the bio-data collected for the certain past time period, calculate a rate of change of the bio-data using the set at least one approximate function, and calculate the bio-data variation using the calculated rate of change.

For example, the computing device 100 may set an approximate function in the form of a quadratic function (e.g., an approximate function in the form of a quadratic function Ax2+Bx+C where the values of the constants A, B, and C are calculated using bio-data at a first time point S, a second time point R, a third time point Q, and a fourth time point P) indicating rates of change at time points (the second time point R, the third time point Q, and the fourth time point P) in three hours before the first time point S.

Subsequently, the computing device 100 may calculate first rates of change which are rates of change at the first time point, the second time point, and the third time point with respect to the first time point and second rates of change which are rates of change at the second time point, the third time point, and the fourth time point with respect to the first time point, and calculate a second bio-data variation p, which is an average variation of the calculated first and second rates of change. However, the present invention is not limited thereto.

According to various embodiments, the computing device 100 may extract the area between one or more different axes on the radial graph as a feature value.

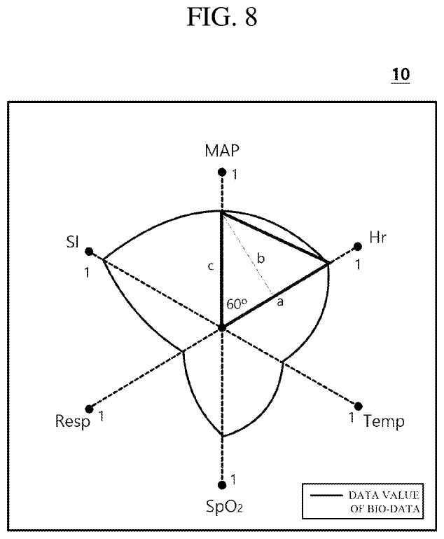

For example, the computing device 100 may extract the area between a first axis representing MAP and a second axis disposed adjacent to the first axis and representing Hr as a feature value. As shown in FIG. 8, when a MAP is c and an HR is a, the area between the first axis and the second axis may be calculated as

a*c*sin(60)/2

Also, the computing device 100 may extract the area between the second axis and a third axis disposed adjacent to the second axis and representing Temp as a feature value.

Also, the computing device 100 may extract the area between the third axis and a fourth axis disposed adjacent to the third axis and representing SpO₂ as a feature value.

Also, the computing device 100 may extract the area between the fourth axis and a fifth axis disposed adjacent to the fourth axis and representing Resp as a feature value.

Also, the computing device 100 may extract the area between the fifth axis and a sixth axis disposed adjacent to the fifth axis and representing SI as a feature value.

Also, the computing device 100 may extract the area between the sixth axis and the first axis disposed adjacent to the fifth axis as a feature value. However, the present invention is not limited thereto, and the computing device 100 may extract the overall area of a closed curve formed on the radial graph as a feature value.

According to various embodiments, the computing device 100 may extract an image of the radial graph as a feature. For example, the computing device 100 may generate an image of the radial graph by capturing the radial graph which is generated as bio-data of the patient is collected, and extract the generated image of the radial graph as a feature. However, the present invention is not limited thereto.

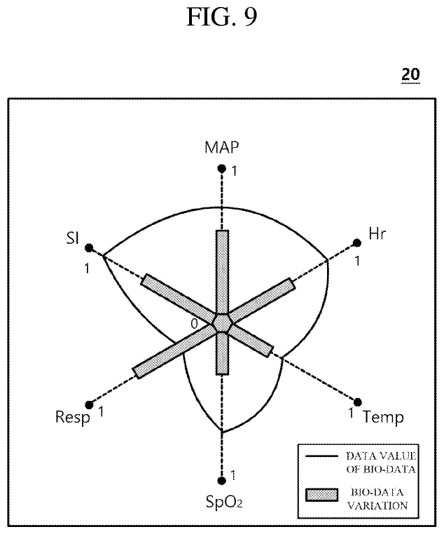

According to various embodiments, the computing device 100 may show the bio-data variations (e.g., calculation of a bio-data variation through Equation 1 and Equation 2 or calculation of a bio-data variation through an approximate function in the form of a quadratic function) calculated in accordance with the above method on the radial graph (e.g., FIG. 9) and the image of the radial graph on which the bio-data variations are shown as a feature. Since each axis of the radial graph has a range of 0 to 1, the bio-data variations may be converted into values within the range of 0 to 1 and then shown on the radial graph.

According to various embodiments, the computing device 100 may extract a color inside the closed curve generated in accordance with the plurality of pieces of data on the radial graph as a feature value. However, the present invention is not limited thereto.

Referring back to FIG. 3, the computing device 100 may determine the probability of the medical event occurring in the patient using the one or more feature values. For example, the computing device 100 may extract result data about the probability of the medical event using the bio-data of the patient and the one or more feature values extracted from the radial graph generated on the basis of the bio-data as input values for an artificial intelligence model and determine the probability of the medical event using the extracted result data.

The artificial intelligence model may include one or more network functions, and the network functions may include a set of calculation units that may be generally referred to as “nodes” and connected to each other. The “nodes” may also be referred to as “neurons.” The one or more network functions may include one or more nodes. The nodes (or neurons) constituting the one or more network functions may be connected to each other through at least one “link.”

In the artificial intelligence model, the one or more nodes connected through the link may have a relative relationship of an input node and an output node. An input node and an output node are relative concepts. Any node which is an output node with respect to one node may be an input node with respect to another node, and vice versa. As described above, the relationship of an input node and an output node may be established on the basis of a link. One or more output nodes may be connected to one input node, and vice versa.

In the relationship of an input node and an output node connected through one link, a value of the output node may be determined on the basis of data input to the input node. A node connecting the input node and the output node to each other may have a weight. The weight may be variable and may be varied by a user or an algorithm to perform a function wanted by the artificial intelligence model. For example, when one or more input nodes are connected to one output node through separate links thereof, the output node may determine an output node value on the basis of values input to the input nodes connected to the output node and weights set for the links each corresponding to the input nodes.

As described above, in an artificial intelligence model, one or more nodes are connected to each other through one or more links and have the relationship of an input node and an output node. A feature of the artificial intelligence model may be determined in accordance with the number of nodes and links and connections between nodes and links in the artificial intelligence model and weights given to the links. For example, when there are two artificial intelligence models in which the same number of nodes and links are present and the corresponding links have different weights, the two artificial intelligence models may be considered to be different.

Some of the nodes constituting the artificial intelligence model may constitute one layer on the basis of distances from an initial input node. For example, a set of nodes having a distance of n from the initial input node may constitute n layer. The distance from the initial input node may be defined by the minimum number of links to be passed through to reach a corresponding node from the initial input node. However, the definition of a layer is arbitrary for description, and the order of a layer in the artificial intelligence model may be defined in a different way than that described above. For example, a layer of nodes may be defined by a distance from a final output node.

The initial input node may be one or more nodes to which data is directly input without passing through a link in the relationship with other nodes among the nodes in the artificial intelligence model. Alternatively, the initial input node may be a node that does not have other input nodes connected through links in the relationship between nodes on the basis of links in the artificial intelligence model network. Similarly, the final output node may be one or more nodes that do not have an output node in the relationship with other nodes among the nodes in the artificial intelligence model. Also, a hidden node may be a node constituting the artificial intelligence model other than the initial input node and the final output node. The artificial intelligence model according to an embodiment of the present disclosure may have a form in which the number of nodes in an input layer may be larger than the number of nodes in a hidden layer and the number of nodes is reduced from the input layer to the hidden layer.

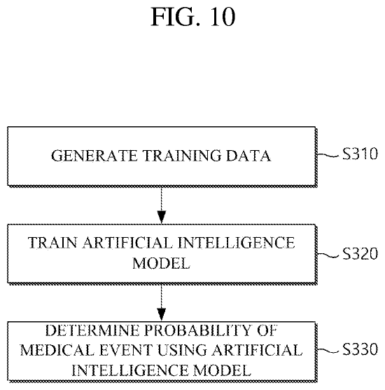

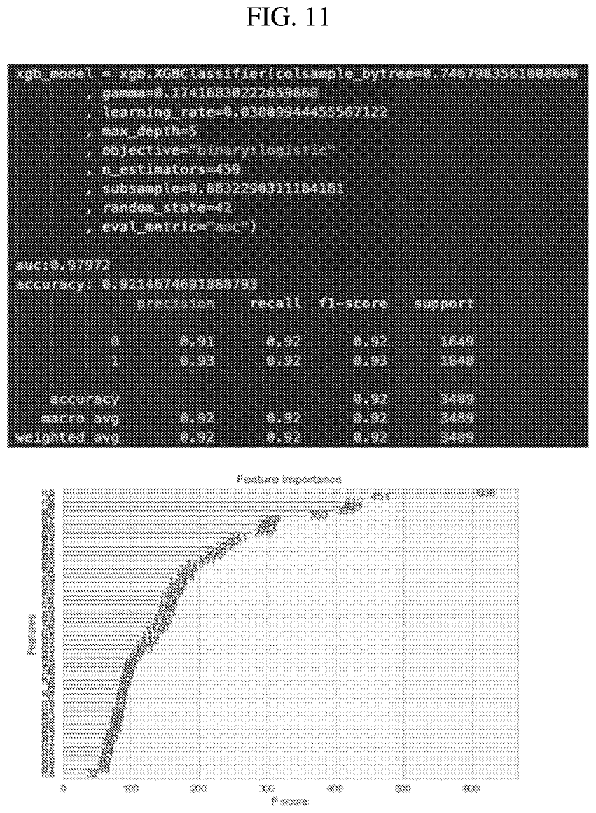

The artificial intelligence model may include one or more hidden layers. Hidden nodes in the hidden layer may use an output of a previous layer and outputs of surrounding hidden nodes as inputs. The hidden layers may have the same or different number of hidden nodes. The number of nodes in the input layer may be determined on the basis of the number of data fields of input data and may be the same as or different from the number of hidden nodes. The input data input to the input layer may be computed by the hidden nodes of the hidden layer and output by a fully connected layer (FCL) which is an output layer. FIGS. 10 and 11 will be described below.

FIG. 10 is a flowchart of a method of training an artificial intelligence model using training data and determining the probability of a medical event using the trained artificial intelligence model according to various embodiments, and FIG. 11 is a diagram illustrating an implementation form of an artificial intelligence model that is applicable to various embodiments.

Referring to FIGS. 10 and 11, in operation S310, the computing device 100 may generate training data for training an artificial intelligence model. For example, the computing device 100 may generate training data by labeling each of bio-data collected at a time point at which a medical event has occurred and bio-data collected for a certain past time period from the time point at which the medical event has occurred among bio-data of a plurality of patients with information about whether the medical event has occurred (e.g., positive—a case in which the medical event has occurred or negative—a case in which the medical event has not occurred), a type of medical event, and time points at which the bio-data has been collected (e.g., one hour before, three hours before, and six hours before). However, the present invention is not limited thereto.

The computing device 100 may provide a user interface (UI) that outputs the bio-data of the patient to the external terminal 200 and obtain a user input for a point at which training data will be generated through the UI. The computing device 100 may generate training data by labeling bio-data corresponding to the user input with information about whether the medical event has occurred, a type of medical event, a time point at which the bio-data has been collected, and a time point at which the medical event has occurred. However, the present invention is not limited thereto, and the computing device 100 may determine whether the medical event has occurred using the bio-data of the patient (e.g., when the MAP is less than 65 or the color inside the closed curve of the radial graph is a color (e.g., black or red) of a time point at which the medical event occurs) and generate training data by automatically labeling patient data of a time point at which the medical event is determined to have occurred.

In operation S320, the computing device 100 may train the artificial intelligence model using the training data generated in operation S310.

More specifically, the computing device may train the one or more network functions constituting the artificial intelligence model using labeled datasets. For example, the computing device 100 may input each of the training input datasets to the one or more network functions and compare each piece of output data calculated through the one or more network functions with each of training output datasets corresponding to labels of the training input datasets to derive errors.

In other words, in the training of the artificial intelligence model, the training input data may be input to input layers of the one or more network functions, and the training output data may be compared with outputs of the one or more network functions. The computing device 100 may train the artificial intelligence model on the basis of the errors between the calculation results of the one or more network functions from the training input data and the training output data (labels).

Also, the computing device 100 may adjust weights of the one or more network functions on the basis of the errors in a backpropagation manner. In other words, the computing device 100 may adjust the weights on the basis of the errors between the calculation results of the one or more network functions from the training input data and the training output data so that outputs of the one or more network functions may approach the training output data.

When the one or more network functions are trained for predetermined epochs or more, the computing device 100 may determine whether to stop training using verification data. The predetermined epochs may be a part of overall training target epochs. The verification data may be at least a part of labeled training datasets. In other words, the computing device 100 may train the artificial intelligence model using the training datasets and determine whether a training effect of the artificial intelligence model is a predetermined level or more using verification data after training of the artificial intelligence model is repeated for the predetermined epochs or more. For example, when training is performed with a target repetitive training number of 10 using 100 pieces of training data, the computing device 100 may repeatedly perform training 10 times which correspond to predetermined epochs and then repeatedly perform training three times using 10 pieces of verification data. When a change in the output of the artificial intelligence model is the predetermined level or less during the three times of repeated training, the computing device 100 may determine that more training is meaningless and finish training. In other words, in repeated training of the artificial intelligence model, verification data may be used for determining completion of training on the basis of whether a training effect of each epoch is a certain level or more. The number of pieces of training data, the number of pieces of verification data, and the number of times repetitive training is performed described above are exemplary, and the present invention is not limited thereto.

The computing device 100 may test performance of the one or more network functions using test datasets and determine whether to activate the one or more network functions, thereby generating the artificial intelligence model. Test data may be used for verifying performance of the artificial intelligence model and may include at least a part of the training datasets. For example, 70% of the training datasets may be used for training the artificial intelligence model (i.e., training for adjusting weights to output a result value similar to the label), and 30% may be used as the test data for verifying performance of the artificial intelligence model.

The computing device 100 may input the test datasets to the artificial intelligence model of which training has been completed, measure an error, and determine whether to activate the artificial intelligence model in accordance with whether the artificial intelligence model shows predetermined performance or more. The computing device 100 may use the test data on the artificial intelligence model of which training has been completed to verify performance of the artificial intelligence model of which training has been completed and when the performance of the artificial intelligence model of which training has been completed is a predetermined reference level or more, may activate the artificial intelligence model so that the artificial intelligence model may be used by other applications.

Also, when the performance of the artificial intelligence model of which training has been completed is the predetermined reference level or less, the computing device 100 may deactivate and discard the artificial intelligence model. For example, the computing device 100 may determine the performance of the generated artificial intelligence model on the basis of factors including accuracy, precision, recall, etc. The foregoing performance criteria are exemplary, and the present invention is not limited thereto.

According to various embodiments, the computing device 100 may generate a plurality of artificial intelligence models by separately training artificial intelligence models, evaluate performance, and use only artificial intelligence models showing certain performance or more to calculate sleep analysis information.

Also, the artificial intelligence model may be trained using at least one of supervised learning, unsupervised learning, and semi-supervised learning. Training of the artificial intelligence model is for minimizing an error of the output. Training of the artificial intelligence model is a process of repeatedly inputting training data to the artificial intelligence model, calculating an error between an output of the artificial intelligence model for the training data and the target, and as a method of reducing the error, backpropagating the error of the artificial intelligence model from the output layer of the artificial intelligence model toward the input layer to update a weight of each node of the artificial intelligence model. In supervised learning, training data labeled with a correct answer (i.e., labeled training data) is used for each piece of training data, and in unsupervised learning, each piece of training data may not be labeled with the correct answer. For example, in the case of supervised learning for data classification, the training data may be data of which each piece is labeled with a category. The labeled training data may be input to the artificial intelligence model, and an error may be calculated by comparing the output (category) of the artificial intelligence model with the label of the training data. As another example, in the case of unsupervised learning for data classification, an error may be calculated by comparing the training data which is an input with the output of the artificial intelligence model. The calculated error is backpropagated in a reverse direction (i.e., from the output layer to the input layer) in the artificial intelligence model, and a connection weight of each node in each layer of the artificial intelligence model may be updated with the backpropagation. A variation of the connection weight of each node to be updated may vary depending on a learning rate. The calculation of the artificial intelligence model for input data and the backpropagation of the error may constitute a learning epoch. The learning rate may be differently applied depending on the repetitive number of learning epochs of the artificial intelligence model. For example, at the beginning of the artificial intelligence model, the artificial intelligence model quickly achieves a certain level of performance using a high learning rate to increase efficiency and at the late stage of learning, a low learning rate is used to increase the accuracy.

In learning of the artificial intelligence model, the training data may generally be a subset of actual data (i.e., data to be processed using the trained artificial intelligence model). Accordingly, in a learning epoch, an error with respect to the training data may be reduced but an error with respect to the actual data may increase. Overfitting is a phenomenon in which training data is excessively learned and thus errors with respect to real data increase. For example, a phenomenon in which an artificial intelligence model that learns cats through yellow cats does not recognize a cat other than yellow cats as a cat may be a sort of overfitting. The overfitting may cause an increase in an error of the machine learning algorithm. To prevent overfitting, a method of increasing training data, regularization, a dropout method of omitting some nodes of a network during a training process, etc. may be used.

In operation S330, the computing device 100 may determine the probability of the event occurring in the patient using the artificial intelligence model trained in operation S320. For example, the computing device 100 may input the one or more feature values extracted by performing the above procedure (e.g., operation S120 of FIG. 3) to the artificial intelligence model as a vector value and extract result data for the input vector value.

According to various embodiments, the artificial intelligence model may include a plurality of artificial intelligence models (e.g., XG Boost (e.g., FIG. 11), a deep learning model, a random forest model, etc.), and the computing device 100 may extract one piece of result data about the probability of the medical event using any one of the plurality of artificial intelligence models and determine the probability of the medical event using the extracted one piece of result data.

According to various embodiments, the computing device 100 may extract two or more pieces of result data about the probability of the medical event using two or more of the plurality of artificial intelligence models and determine the probability of the medical event by aggregating the extracted two or more pieces of result data. For example, the computing device 100 may calculate the summation of the result data of the two or more artificial intelligence models and input the summation to an artificial intelligence model for a final determination, thereby determining the probability of the medical event.

In the disclosed embodiments, a method of collecting bio-data of a patient using an apparatus for measuring and collecting bio-data of a patient and analyzing the collected bio-data to predict whether the patient will develop a shock has been described. According to an exemplary embodiment, the apparatus for measuring and collecting bio-data of a patient may be a patient monitor used in a hospital. However, a type of apparatus according to the disclosed embodiments is not limited thereto, and in addition to the patient monitor, other apparatus may be used for collecting bio-data of a patient.

Further, the technology according to the disclosed embodiments may be used for measuring and collecting bio-data of a patient in the patient's daily life outside a hospital and predicting the patient's shock or abnormal condition on the basis of the bio-data.

According to various embodiments, the computing device 100 may collect bio-data of a patient that is measured through a mobile terminal (e.g., a wearable device such as a smart watch) worn on at least a part of the patient's body rather than a bedside monitoring device in an intensive care unit.

Types of bio-data measured through the bed-side monitoring device in an intensive care unit may be SI, Resp, SO₂, Temp, Hr, and MAP, and types of bio-data measured through the wearable device may be blood pressure, pulse, oxygen saturation (SpO₂), and electrocardiogram (ECG) values. However, types of bio-data are not limited thereto.

For example, types of data that are collectable through the wearable device may be limited compared to types of data that are collectable through the patient monitor. Accordingly, in this case, the patient's shock may be predicted by performing the analysis method according to the disclosed embodiment on the basis of some data that is collectable through the wearable device.

According to various embodiments, when blood pressure, pulse, oxygen saturation (SpO₂), and ECG values are collected through the wearable device, the computing device 100 may convert the collected values into values within the range of 0 to 1 using a method identical or similar to the method described in the disclosed embodiment (e.g., any one of approximate function estimation with respect to a normal distribution and approximate function estimation with respect to a probability distribution).

According to various embodiments, when blood pressure, pulse, oxygen saturation (SpO₂), and ECG values are collected through the wearable device, the computing device 100 may set the blood pressure, pulse, oxygen saturation, and ECG values as axes and set the angle between the axes to 90° so that the four axes may have the same angle therebetween. However, the angle between axes is not limited thereto. When the number of pieces of available data is limited, axes representing the data may be arranged so that the available data may have the same angular intervals therebetween, and the data may be visualized on the basis of the arrangement.

Subsequently, the computing device 100 may generate a radial graph having four axes (e.g., an operation of reversing the value of each axis, an operation of generating a closed curve, and an operation of determining a color inside the closed curve) using a method identical or similar to the above method (e.g., the method of generating a radial graph having six axes using six types of bio-data).

When the patient's shock is predicted on the basis of the information collected through the wearable device, the computing device 100 may transmit the corresponding information to an external institution such as a hospital and the like. In this case, the external institution, such as the hospital and the like may contact the patient so that the patient may visit the hospital, or may send an ambulance to transfer the patient to the hospital. Also, a process in which a medical expert reviews the transmitted information to determine whether there is the probability of an actual emergency may be performed.

There is no limitation on the type of wearable device according to the disclosed embodiment, and instead of the wearable device, a device in the form of various tools used in daily life and including one or more sensors may be used according to an embodiment.

For example, one or more sensors connected to the electronic system of a vehicle may be used, and the sensors may be provided in at least one of the steering wheel and the gear knob of the vehicle. For example, the sensors may be provided at two different points in the steering wheel and the gear knob of the vehicle. When a user grabs sensor portions at the two different points of the steering wheel or grabs the steering wheel with one hand and grabs the gear knob with the other hand, a circuit including the user's body is constructed and used for measuring the user's pulse and ECG.

In addition, the user's respiration, blood pressure, oxygen saturation, etc. may be measured using a sensor device provided in the vehicle, but the present invention is not limited thereto. The sensor device provided in the vehicle may be used together with a sensor device provided in the wearable device.

In this way, it is possible to predict that a shock or an abnormal condition will occur in the user during driving. In this case, the user may be induced to stop driving and call an ambulance, or when enough time remains until the shock occurs and there is a nearby hospital, the user may be induced to immediately move to the hospital.

According to another embodiment, when it is determined during driving that a shock has occurred to the user or there is the probability of a shock occurring in the user, a driving mode may be switched from manual driving to an autonomous driving mode. In this case, the vehicle may move to the closest hospital or emergency room through autonomous driving and automatically provide a notification to the medical institution.

The method of predicting a patient's shock using artificial intelligence has been described above with reference to the flowcharts shown in the drawings. While the method of predicting a patient's shock using artificial intelligence has been shown and described as a series of blocks for the purpose of simplicity of explanation, the present invention is not limited to the order of the blocks. Some blocks may be performed concurrently or in an order different from that shown and described herein. Also, a new block which is not shown in the drawings may be added, or some blocks may be removed or changed.

Although embodiments of the present invention have been described with reference to the accompanying drawings, those skilled in the technical field to which the present invention pertains should appreciate that the present invention can be implemented in other specific forms without changing the technical spirit or essential characteristics. Therefore, the above-described embodiments should be understood as illustrative and not restrictive in all aspects.

1. A method of predicting a patient's shock using artificial intelligence by a computing device, the method comprising:
collecting bio-data of a patient;
extracting one or more feature values from the collected bio-data; and
determining a probability of a medical event occurring in the patient using the extracted one or more feature values.

2. The method of claim 1, wherein the collecting of the bio-data comprises collecting a plurality of pieces of bio-data including at least one of a shock index (SI), respiration (Rr), saturation of percutaneous oxygen (SpO₂), a temperature (Temp), a heart rate (Hr), and a mean arterial pressure (MAP) of the patient, and
the extracting of the one or more feature values comprises:
converting each of the plurality of pieces of bio-data into a value within a preset range;
generating a radial graph in which each individual axis corresponds to one of the plurality of pieces of bio-data converted into the value within the preset range; and
extracting the one or more feature values using the generated radial graph.

3. The method of claim 2, wherein the converting of each of the plurality of pieces of bio-data into the value within the preset range comprises:
converting the SI and the Temp of the patient into values within a range of 0 to 1 using SIs and Temps obtained by transforming SIs and Temps of a plurality of patients into a normal distribution; and
calculating an approximate function for each of Rr, SpO₂, Hr, and MAP of each of the plurality of patients using Bayesian probability distributions of Rr, SpO₂, Hr, and MAP when the medical event occurs in the plurality of patients and converting each of the SO₂, the Hr, and the MAP of the patient into a value within a range of 0 to 1 using the calculated approximate function.

4. The method of claim 2, wherein the extracting of the one or more feature values using the generated radial graph comprises:
extracting a data value of bio-data collected at a first time point as a feature value;
extracting a bio-data variation which is a difference value between the data value of the bio-data collected at the first time point and a data value of bio-data collected for a certain past time period from the first time point as a feature value; and
extracting an area between one or more different axes in the generated radial graph as a feature value.

5. The method of claim 4, wherein the extracting of the area between the one or more different axes in the generated radial graph as the feature value comprises extracting, as feature values, an area between a first axis representing MAP and a second axis disposed adjacent to the first axis and representing Hr,
an area between the second axis and a third axis disposed adjacent to the second axis and representing Temp,
an area between the third axis and a fourth axis disposed adjacent to the third axis and representing SpO₂,
an area between the fourth axis and a fifth axis disposed adjacent to the fourth axis and representing Rr,
an area between the fifth axis and a sixth axis disposed adjacent to the fifth axis and representing SI, and
an area between the sixth axis and the first axis disposed adjacent to the six axis.

6. The method of claim 4, wherein the extracting of the bio-data variation as the feature value comprises:
determining the first time point and a plurality of past time points preceding the first time point;
determining a plurality of combination pairs of time points including the first time point and the plurality of past time points;
calculating a variation of bio-data values corresponding to time points included in each of the plurality of combination pairs; and
calculating a feature value of the bio-data variation using the variations each calculated from the plurality of combination pairs.

7. The method of claim 4, wherein the extracting of the bio-data variation as the feature value comprises:
setting an approximate function which is a quadratic function using the data value of the bio-data collected at the first time point and the data value of the bio-data collected for the certain past time period; and
calculating a rate of change of the bio-data using the set at least one approximate function and calculating the bio-data variation using the calculated rate of change.

8. The method of claim 2, wherein the extracting of the one or more feature values using the generated radial graph comprises extracting an image of the generated radial graph as a feature, and
the determining of the probability of the medical event comprises inputting the image of the radial graph to a trained model to determine the probability of the medical event on the basis of an output of the trained model.

9. The method of claim 8, wherein the extracting of the image of the generated radial graph as the feature comprises:
calculating a bio-data variation which is a difference value between a data value of bio-data collected at a first time point and a data value of bio-data collected for a certain past time period from the first time point;
showing the calculated bio-data variation on the generated radial graph; and
extracting an image of the radial graph showing the calculated bio-data variation as a feature.

10. The method of claim 2, wherein the generating of the radial graph comprises connecting each of the SI, the Rr, the SpO₂, the Temp, the Hr, and the MAP to the adjacent bio-data to form a closed curve and determining a color inside the formed closed curve in accordance with an area inside the formed closed curved, and
the extracting of the one or more feature values using the generated radial graph comprises extracting the determined color inside the closed curve as a feature value.

11. The method of claim 1, wherein the determining of the probability of the medical event comprises:
training an artificial intelligence model using bio-data of a plurality of patients as training data; and
extracting result data about the probability of the medical event using the extracted one or more feature values as input values for the trained artificial intelligence model and determining the probability of the medical event using the extracted result data, and
wherein the training of the artificial intelligence model comprises:
labeling each of bio-data collected at a time point at which the medical event has occurred and bio-data collected for a certain past time period from the time point at which the medical event has occurred among the bio-data of the plurality of patients with information about whether the medical event has occurred, a type of medical event, and a time point at which the bio-data has been collected to generate the training data; and
training the artificial intelligence model in accordance with supervised learning using the generated training data.

12. The method of claim 11, wherein the artificial intelligence model includes a plurality of artificial intelligence models, and
the determining of the probability of the medical event using the extracted result data comprises:
extracting one piece of result data about the probability of the medical event using any one of the plurality of artificial intelligence models and determining the probability of the medical event using the extracted one piece of result data; or
extracting two or more pieces of result data about the probability of the medical event using two or more of the plurality of artificial intelligence models and aggregating the extracted two or more pieces of result data to determine the probability of the medical event.

13. A device for predicting a patient's shock using artificial intelligence, the device comprising:
a processor;
a network interface;
a memory; and
a computer program loaded into the memory and executed by the processor,
wherein the computer program comprises:
an instruction for collecting bio-data of a patient;
an instruction for extracting one or more feature values from the collected bio-data; and
an instruction for determining a probability of a medical event occurring in the patient using the extracted one or more feature values.

14. A computer program recorded on a computer-readable recording medium to perform, in combination with a computing device:
collecting bio-data of a patient;
extracting one or more feature values from the collected bio-data; and
determining a probability of a medical event occurring in the patient using the extracted one or more feature values.
